(19) United States
(12) Patent Application Publication
(10) Pub. No.: US 2016/0251387 A1
(43) Pub. Date: Sep. 1, 2016

(54) SYNTHESIS OF 1-ALKYL-2-AMINO-IMIDAZOL-5-CARBOXYLIC ACID ESTER VIA CALPHA-SUBSTITUTED N-ALKYL-GLYCINE ESTER DERIVATIVES

(71) Applicant: MERCK PATENT GMBH, Darmstadt (DE)

(72) Inventors: Christian JASPER, Seligenstadt (DE); Helmut Dieter HAHN, Ruesselsheim (DE); Marcel Andre BREUNING, Frankfurt am Main (DE); Philipp KRATTIGER, Darmstadt (DE); Michaela DEBOR, Stockstadt (DE)

(21) Appl. No.: 15/028,200

(22) PCT Filed: Oct. 10, 2014
(86) PCT No.: PCT/EP2014/002751

§ 371 Date: Apr. 8, 2016

Related U.S. Application Data
(60) Provisional application No. 61/889,256, filed on Oct. 10, 2013.

(51) Int. Cl. C07F 9/6506; C07D 317/30

(52) U.S. Cl. CPC C07F 9/65065; C07D 317/30

(57) ABSTRACT

The invention provides an efficient and high yielding process for preparing TH-302, comprising at least one step wherein a dioxolane intermediate is generated in an aqueous layer, resulting in a synthesis that is amenable to scale up conditions.

### BACKGROUND OF THE INVENTION

Phosphoramidate based alkylators used in cancer therapy, such as Cyclophosphamide and Ifosfamide, are an important subclass of chemotherapeutic alkylators. Cyclophosphamide and Ifosfamide are each activated in the liver and the active alkylator released alkylates nucleophilic moieties such as the DNA within the tumor cells to act as a chemotherapeutic agent. If the active alkylators are released away from the tumor, DNA and other nucleophilic moieties such as the phosphate, amino, sulfhydryl, hydroxyl, carboxyl and imidazo groups of biomolecules of healthy non-cancerous cells, can get alkylated. Such alkylation of healthy cells can result in unwanted toxic events in patients (see Hardman et al., supra).

There remains a need for new phosphoramidate based alkylators that can be used to treat cancer or other hyperproliferative disease conditions, preferably compounds less toxic to normal cells. TH-302 is such a compound, and is described in WO 07/002931. The present invention is directed towards novel methods of producing TH-302 and novel methods of producing novel intermediates.

### SUMMARY OF THE INVENTION

In certain aspects, the invention is directed towards an efficient and high yielding process for preparing TH-302:

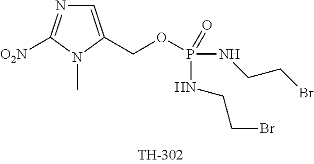 [SMILES: Cn1c(COP(=O)(NCCBr)NCCBr)cnc1[N+](=O)[O-]]

or a pharmaceutically acceptable salt thereof.

In certain embodiments, the invention provides a method of making TI-1-302, or a pharmaceutically acceptable salt thereof, comprising the step of converting a compound or salt of formula V

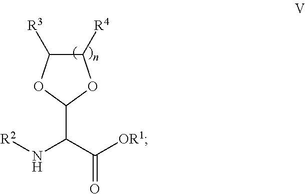

wherein R¹, R², R³, R⁴, and n are as described below,
to a compound or salt of formula IV

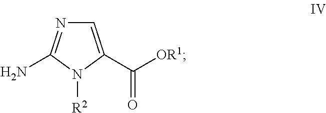

and converting the compound or salt of formula IV to TH-302, or a salt thereof.

### DETAILED DESCRIPTION OF THE INVENTION

### 1. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.,

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

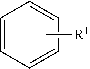

refers to at least

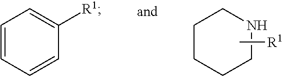

refers to at least

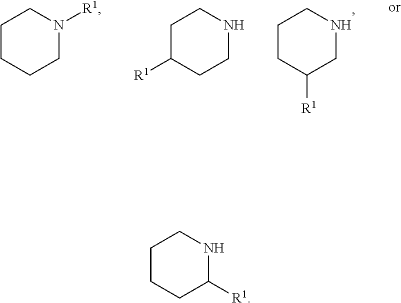

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which are optionally substituted with R^{∘}; —(CH₂)₀₋₄O(CH₂)₀₋₁Ph which is optionally substituted with R^{∘}; —CH═CHPh, which is optionally substituted with R^{∘}; —(CH₂)₀₋₄O(CH₂)₀₋₁-pyridyl which is optionally substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄S(O)₂R; —S(O)₂NR^{∘}₂; —(CH₂)₀₋₄S(O)R; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} is optionally substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{∘} (or the ring formed by taking two independent occurrences of R^{∘} together with their intervening atoms), are independently deuterium, halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)O₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)O₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{} include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR¹, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:

—F, —Cl, —Br, —I, deuterium,

—OH, protected hydroxy, alkoxy, oxo, thiooxo,

—NO₂, —CN, CF₃, N₃,

—NH₂, protected amino, —NH alkyl, —NH alkenyl, —NH alkynyl, —NH cycloalkyl, —NH -aryl, —NH-heteroaryl, —NH-heterocyclic, -dialkylamino, -diarylamino, -diheteroarylamino,

—O— alkyl, —O— alkenyl, —O— alkynyl, —O— cycloalkyl, —O-aryl, —O-heteroaryl, —O-heterocyclic,

—C(O)— alkyl, —C(O)— alkenyl, —C(O)— alkynyl, —C(O)— carbocyclyl, —C(O)-aryl, —C(O)-heteroaryl, —C(O)-heterocyclyl,

—CONH₂, —CONH— alkyl, —CONH— alkenyl, —CONH— alkynyl, —CONH— carbocyclyl, —CONH-aryl, —CONH-heteroaryl, —CONH-heterocyclyl,

—OCO₂— alkyl, —OCO₂— alkenyl, —OCO₂— alkynyl, —OCO₂— carbocyclyl, —OCO₂-aryl, —OCO₂-heteroaryl, —OCO₂-heterocyclyl, —OCONH₂, —OCONH— alkyl, —OCONH— alkenyl, —OCONH— alkynyl, —OCONH— carbocyclyl, —OCONH-aryl, —OCONH— heteroaryl, —OCONH— heterocyclyl,

—NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂— alkyl, —NHCO₂— alkenyl, —NHCO₂— alkynyl, —NHCO₂— carbocyclyl, —NHCO₂-aryl, —NHCO₂— heteroaryl, —NHCO₂— heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH— heteroaryl, —NHC(O)NH-heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH- -alkenyl, —NHC(NH)NH— alkenyl, —NHC(NH)NH— carbocyclyl, —NHC(NH)NH-aryl, —NHC(NH)NH-heteroaryl, —NHC(NH)NH-heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl,

—C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl,

—S(O)— alkyl, —S(O)— alkenyl, —S(O)— alkynyl, —S(O)— carbocyclyl, —S(O)-aryl, —S(O)-heteroaryl, —S(O)-heterocyclyl —SO₂NH₂, —SO₂NH— alkyl, —SO₂NH— alkenyl, —SO₂NH— alkynyl, —SO₂NH— carbocyclyl, —SO₂NH— aryl, —SO₂NH— heteroaryl, —SO₂NH— heterocyclyl,

—NHSO₂— alkyl, —NHSO₂— alkenyl, —NHSO₂— alkynyl, —NHSO₂— carbocyclyl, —NHSO₂-aryl, —NHSO₂-heteroaryl, —NHSO₂-heterocyclyl,

—CH₂NH₂, —CH₂SO₂CH₃,

-mono-, di-, or tri-alkyl silyl,

-alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

There is furthermore intended that a compound of the invention includes isotope-labeled forms thereof. An isotope-labeled form of a compound of the invention is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the invention by well-known methods include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶CI, respectively. A compound of the invention, a prodrug, thereof or a pharmaceutically acceptable salt of either which contains one or more of the above-mentioned isotopes and/or other isotopes of other atoms is intended to be part of the present invention.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### 2. Overview of Syntheses of the Invention

A method of synthesis of the intermediates of the invention, and derivatives thereof, shown below in Scheme 1, has been previously reported in the literature (G. Asato, G. Berkelhammer, J. Med. Chem. 1972, 1086; U.S. Pat. No. 2,516,900A). The methods described in the art are not applicable for larger scales, due to the use of benzene and the addition of strong bases such as NaOMe or NaOEt in neat form. Additionally, the isolation of Na-enolate salt intermediates is not preferable due to air and moisture-sensitivity issues.

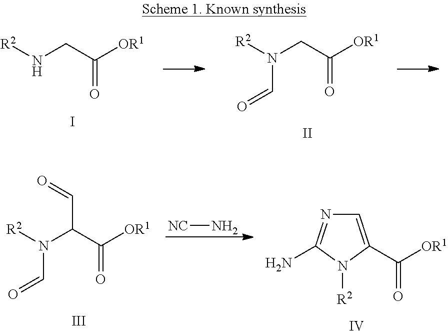

In Scheme 1, deprotection of III is carried out with HCl in MeOH, resulting the secondary amine as its HCl salt, while in the presence of the C-formyl group. Dimerization/oligomerization reactions between the amine and the aldehyde are believed to be responsible for the low yields (45%) reported by Asato. Further, four filtration steps are necessary to isolate the desired compound IV as the free amine, starting from III.

Conversion of the compounds of formula IV to TH-302 was carried out by the methods described in WO 07/002931 and US 2011/0251159.

In certain aspects, the current invention is directed towards a more efficient synthesis of compound IV, without byproduct formation due to dimerization or oligomerization side reactions starting with compound III. The current invention is also directed towards a reduction in the number of filtration steps and solvent changes, allowing for a large scale synthesis of compound IV in high yield with less solvent changes. The reaction sequence is described in Scheme 2.

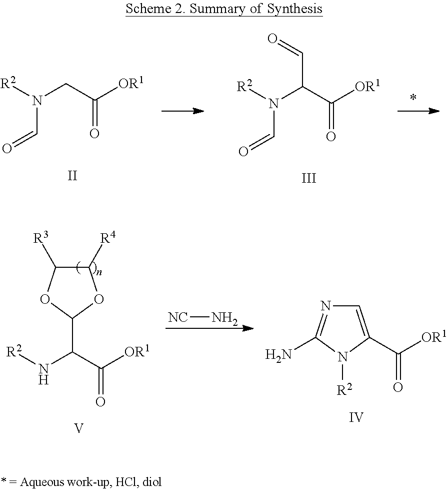

The procedure of starts from the N-formylsarcosine ethyl ester of formula II and uses a base, such as potassium tert-butylate as base in tetrahydrofurane (20 wt-%) to synthesize a compound of formula III, or an enolate of III.

The use of a base in solution allows for much better reagent control for larger scales. Moreover, the enolate of formula III is not isolated as a precipitate from organic solvents, but is extracted from the reaction mixture with water. This approach was developed by Jones for 2-mercaptoimidazole derivatives using KSCN as reagent and was intensively modified here to be applicable for the synthesis of 2-aminoimidazole derivatives using cyanamide (CN—NH₂) as a reagent.

A diol (e.g. Ethylene glycol, propylene glycol) is used as a water soluble protecting group for the C-formyl functionality, and the conversion of the HCl-water phase to a HOAc/NaOAc buffer system via addition of NaOAc, was used instead of first removing the HCl by distillation. As a result, the aqueous extracts are directly processed and avoids the isolation of neat V, while the buffer system for the final conversion of V to IV is directly set up (important due to cyanamide stability: “CN—NH₂ has the highest stability in aqueous sol. at a pH of 4-4.5, while strong mineralic acids catalyze the hydrolysis to urea”; source: chapter “Cyanamides” in Ullmann's Encyclopedia of Industrial Chemistry, Wiley-VCH 2012, Weinheim, Germany).

In various embodiments, the compounds of formula IV are converted to TH-302 using methods known in the art.

The advantages of the methods of this invention are at least as follows: a) easy preparation of III or its enolate was possible without being isolated; b) byproduct formation was reduced, and the yield was improved from 45% to 75%; c) only 1 filtration step was necessary, which is the product isolation at the end.

### 3. Description of Certain Embodiments of the Invention

The subject invention will now be described in terms of certain embodiments. These embodiments are set forth to aid in understanding the invention but are not to be construed as limiting.

In one aspect, the invention is directed towards an efficient and high yielding process for preparing TH-302:

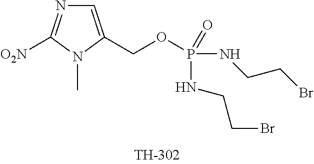 [SMILES: Cn1c(COP(=O)(NCCBr)NCCBr)cnc1[N+](=O)[O-]]

or a pharmaceutically acceptable salt thereof.

In certain embodiments, the invention provides a method of making TH-302, comprising the step of converting a compound of formula V

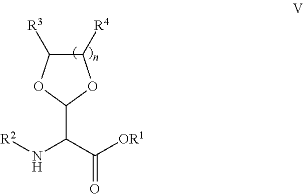

or a salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
   n is 0, 1, 2, or 3;
   to a compound of formula IV

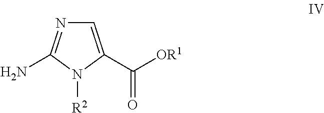

or a salt thereof,
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
   and converting the compound of formula IV to TH-302, or a pharmaceutically acceptable salt thereof.

In certain embodiments, the invention provides a method of making TH-302, or a pharmaceutically acceptable salt thereof, comprising the step of converting a compound of formula III

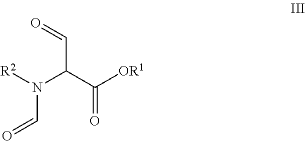

or a salt thereof,
wherein R¹ and R² are as described previously, to a compound of formula V

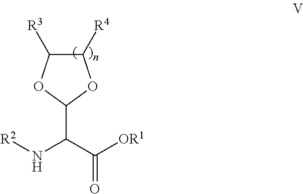

or a salt thereof,
wherein R¹, R², R³, R⁴, and n are as described previously,
and converting the compound of formula V to TH-302, or a pharmaceutically acceptable salt thereof.

In certain embodiments, the invention provides a method of making TH-302, or a pharmaceutically acceptable salt thereof, comprising the step of converting a compound of formula II

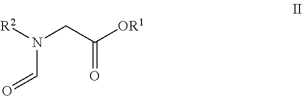

or a salt thereof,
wherein R¹ and R² are as described previously, to a compound of formula III

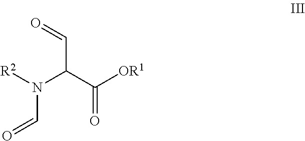

or a salt thereof,
and converting the compound of formula III to TH-302, or a pharmaceutically acceptable salt thereof.

In various embodiments, compounds of formula V are in an aqueous solvent.

In various embodiments, compounds of formula III, or enolate thereof, are extracted into an aqueous medium. In various embodiments, compounds of formula III, which were extracted into an aqueous medium, are converted to compounds of formula V in an aqueous medium.

In certain embodiments, the invention provides a compound of any formulae presented herein wherein R¹ is C₁₋₆ aliphatic which is optionally substituted. In certain embodiments, the invention provides a compound of any formulae presented herein wherein R¹ is a 3-8 membered saturated or partially unsaturated carbocyclic ring which is optionally substituted. In certain embodiments, the invention provides a compound of any formulae presented herein wherein R¹ is a 3-7 membered heterocylic ring having 1-4 heteroatoms independently selected from nitrogen, oxygen, or sulfur, which is optionally substituted. In certain embodiments, the invention provides a compound of any formulae presented herein wherein R¹ is a 5-6 membered monocyclic heteroaryl ring having 1-4 heteroatoms independently selected from nitrogen, oxygen, or sulfur, which is optionally substituted.

In certain embodiments, R¹ is methyl, ethyl, propyl, i-propyl, butyl, s-butyl, t-butyl, straight or branched pentyl, or straight or branched hexyl; each of which is optionally substituted. In certain embodiments, R¹ is methyl or ethyl.

In certain embodiments, R¹ is phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, the invention provides a compound of any formulae presented herein wherein R² is C₁₋₆ aliphatic which is optionally substituted. In certain embodiments, the invention provides a compound of any formulae presented herein wherein R² is a 3-8 membered saturated or partially unsaturated carbocyclic ring which is optionally substituted. In certain embodiments, the invention provides a compound of any formulae presented herein wherein R² is a 3-7 membered heterocylic ring having 1-4 heteroatoms independently selected from nitrogen, oxygen, or sulfur, which is optionally substituted. In certain embodiments, the invention provides a compound of any formulae presented herein wherein R² is a 5-6 membered monocyclic heteroaryl ring having 1-4 heteroatoms independently selected from nitrogen, oxygen, or sulfur, which is optionally substituted.

In certain embodiments, R² is methyl, ethyl, propyl, i-propyl, butyl, s-butyl, t-butyl, straight or branched pentyl, or straight or branched hexyl; each of which is optionally substituted. In certain embodiments, R² is methyl or ethyl.

In certain embodiments, R² is phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, the invention provides a compound of any formulae presented herein, wherein R² is —SO₂R¹. In certain embodiments, the invention provides a compound of any formulae presented herein, wherein R² is —SOR¹. In certain embodiments, the invention provides a compound of any formulae presented herein, wherein R² is —C(O)R¹. In certain embodiments, the invention provides a compound of any formulae presented herein, wherein R² is —CO₂R¹. In certain embodiments, the invention provides a compound of any formulae presented herein, wherein R² is —C(O)N(R¹)₂.

In various embodiments, the invention provides a compound of any formulae presented herein wherein R³ is hydrogen.

In certain embodiments, the invention provides a compound of any formulae presented herein wherein R³ is C₁₋₆ aliphatic which is optionally substituted. In certain embodiments, the invention provides a compound of any formulae presented herein wherein R³ is a 3-8 membered saturated or partially unsaturated carbocyclic ring which is optionally substituted. In certain embodiments, the invention provides a compound of any formulae presented herein wherein R³ is a 3-7 membered heterocylic ring having 1-4 heteroatoms independently selected from nitrogen, oxygen, or sulfur, which is optionally substituted. In certain embodiments, the invention provides a compound of any formulae presented herein wherein R³ is a 5-6 membered monocyclic heteroaryl ring having 1-4 heteroatoms independently selected from nitrogen, oxygen, or sulfur, which is optionally substituted.

In certain embodiments, R³ is methyl, ethyl, propyl, i-propyl, butyl, s-butyl, t-butyl, straight or branched pentyl, or straight or branched hexyl; each of which is optionally substituted. In certain embodiments, R³ is methyl or ethyl.

In various embodiments, the invention provides a compound of any formulae presented herein wherein R³ is halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In various embodiments, the invention provides a compound of any formulae presented herein wherein R⁴ is hydrogen.

In certain embodiments, the invention provides a compound of any formulae presented herein wherein R⁴ is C₁₋₆ aliphatic which is optionally substituted. In certain embodiments, the invention provides a compound of any formulae presented herein wherein R⁴ is a 3-8 membered saturated or partially unsaturated carbocyclic ring which is optionally substituted. In certain embodiments, the invention provides a compound of any formulae presented herein wherein R⁴ is a 3-7 membered heterocylic ring having 1-4 heteroatoms independently selected from nitrogen, oxygen, or sulfur, which is optionally substituted. In certain embodiments, the invention provides a compound of any formulae presented herein wherein R⁴ is a 5-6 membered monocyclic heteroaryl ring having 1-4 heteroatoms independently selected from nitrogen, oxygen, or sulfur, which is optionally substituted.

In certain embodiments, R⁴ is methyl, ethyl, propyl, i-propyl, butyl, s-butyl, t-butyl, straight or branched pentyl, or straight or branched hexyl; each of which is optionally substituted. In certain embodiments, R⁴ is methyl or ethyl.

In various embodiments, the invention provides a compound of any formulae presented herein wherein R⁴ is halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In various embodiments, the invention provides a compound of any formulae presented herein wherein n is 1. In various embodiments, the invention provides a compound of any formulae presented herein wherein n is 2.

In certain embodiments, the invention is directed towards an efficient and high yielding process for preparing TH-302:

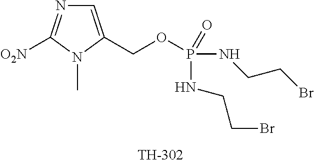 [SMILES: Cn1c(COP(=O)(NCCBr)NCCBr)cnc1[N+](=O)[O-]]

or a pharmaceutically acceptable salt thereof,
comprising the step of converting a compound of formula III

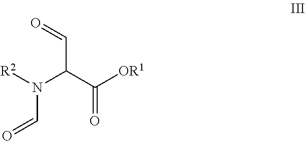

or a salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
R² is —R¹, -haloalkyl, —SO₂R¹, —SOR¹, —C(O)R¹, —CO₂R¹, or —C(O)N(R¹)₂,
   to a compound of formula V

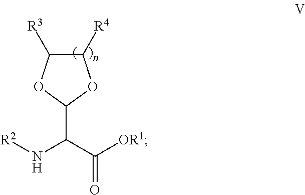

or a salt thereof,
wherein R¹, R², R³, R⁴, and n are as described previously, converting the compound of formula V to a compound of formula IV

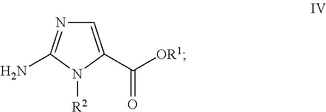

or a salt thereof,
wherein R¹ and R² are as described previously,
and converting the compound of formula IV to TH-302, or a pharmaceutically acceptable salt thereof.

In certain embodiments, the compound of formula V is in an aqueous solvent.

In certain embodiments, the compound of formula III, or enolate thereof, is extracted into an aqueous medium. In various embodiments, compounds of formula III, which were extracted into an aqueous medium, are converted to compounds of formula V in an aqueous medium.

In one aspect, the invention is directed towards an efficient and high yielding process for preparing compounds of formula IV:

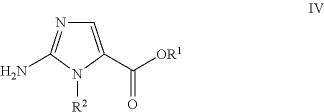

or a pharmaceutically acceptable salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
R² is —R¹, -haloalkyl, —SO₂R¹, —SOR¹, —C(O)R¹, —CO₂R¹, or —C(O)N(R¹)₂.

In certain embodiments, the invention provides a method of making a compound of formula IV, or a pharmaceutically acceptable salt thereof, comprising the step of converting a compound of formula V

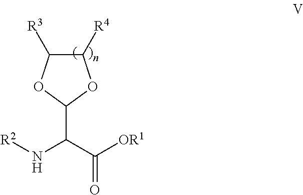

or a salt thereof,
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
   n is 0, 1, 2, or 3;
   to a compound of formula IV

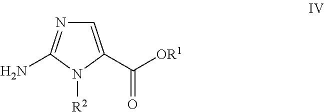

or a salt thereof.

In certain embodiments, the invention provides a method of making a compound of formula IV, comprising the step of converting a compound of formula III

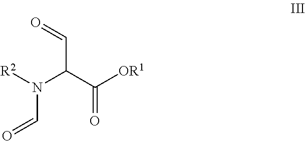

or a salt thereof,
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
   to a compound of formula V

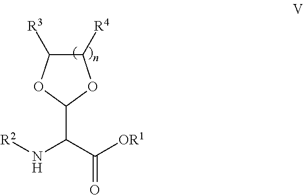

wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
   n is 0, 1, 2, or 3;
   or a salt thereof.

In certain embodiments, the invention provides a method of making a compound of formula IV, or a salt thereof, comprising the step of converting a compound of formula II

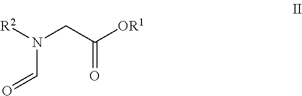

or a salt thereof,
wherein R¹ and R² are as described previously,
to a compound of formula III

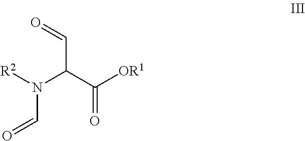

or a salt thereof,
wherein R¹ and R² are as described previously.

In certain embodiments, the compound of formula V is in an aqueous solvent.

In certain embodiments, the compound of formula III, or enolate thereof, is extracted into an aqueous medium. In various embodiments, compounds of formula III, which were extracted into an aqueous medium, are converted to compounds of formula V in an aqueous medium.

In certain embodiments, the invention is directed towards an efficient and high yielding process for preparing a compound of formula IV:

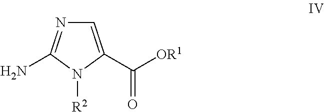

or a salt thereof,
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
   comprising the step of converting a compound of formula III

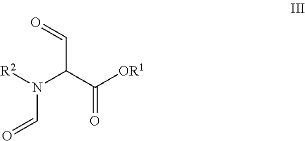

or a salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
R² is —R¹, -haloalkyl, —SO₂R¹, —SOR¹, —C(O)R¹, —CO₂R¹, or —C(O)N(R¹)₂, to a compound of formula V

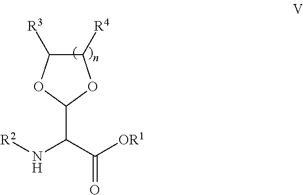

or a salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
   n is 0, 1, 2, or 3;
   and converting the compound of formula V to a compound of formula IV

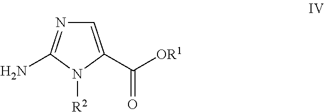

or a salt thereof.

In certain embodiments, the above compounds of any of the formulae above, include pharmaceutically acceptable salts thereof. In certain embodiments, the above compounds of any of the formulae above, include salts thereof. In certain embodiments, the above compounds of any of the formulae above, include acid salts thereof. In certain embodiments, the above compounds of any of the formulae above, include base salts thereof.

In certain embodiments, the compound of formula V is in an aqueous solvent.

In certain embodiments, the compound of formula III, or enolate thereof, is extracted into an aqueous medium. In various embodiments, compounds of formula III, which were extracted into an aqueous medium, are converted to compounds of formula V in an aqueous medium.

In certain embodiments, the invention contemplates a method as described above, wherein the compound or intermediate of formula III is an enolate thereof, including

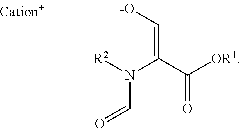

In certain embodiments, the invention provides a method, wherein the conversion of II to III, or enolate thereof, comprises a base, a formyl source such as ethyl formate (e.g. alkyl formates, N-Formylpiperidin, N-Formylmorpholin), and one or more solvents. Bases are any basic chemical, which can be inorganic (e.g., sodium bicarbonate, potassium hydroxide), or organic (e.g., triethylamine, pyridine) or organic salts (e.g. n-Alkyllithium, Lithium diisopropylamide, hexamethyldisilazid bases) in nature. Bases are useful in either catalytic or stoichiometric amounts to facilitate chemical reactions. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. In certain embodiments, the base is KOtBu. In certain embodiments, the solvent is selected from one or more of THF, Methyl-THF, toluene, xylene, ether, MTBE, cumene, aliphatic hydrocarbons or methylene chloride.

In certain embodiments, the invention provides a method, wherein the conversion of III, or enolate thereof, to V comprises an aqueous extraction of III followed by addition of a water soluble diol (e.g. ethylene glycol or propylene glycol), to provide V in the aqueous layer. In certain embodiments, the conversion of III, or enolate thereof, to V further comprises the addition of HCl to the aqueous layer.

In certain embodiments, the invention provides a method, wherein the conversion of V to IV comprises a water soluble base and NC—NH₂. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. In certain embodiments, the base is NaOAc.

In certain embodiments of the reactions provided above, an acid may be used.

Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid. In another embodiment, the invention provides a method, wherein the Lewis Acid is, e.g., aluminum chloride, diethylaluminum chloride, or ethylaluminium dichloride.

In certain embodiments, the methods presented above provide a step of adding an acid before or during the addition of cyanamide, to provide stability to the cyanamide. In certain embodiments, an acid is added so that the aqueous solution has a pH of about 3-6.5. In certain embodiments, the pH is about 4-4.5.

In certain embodiments, the conversion from II to III takes place from about 0° C. to about 25° C. In certain embodiments, the conversion from II to III takes place from about 0° C. to about 10° C. In certain embodiments, the conversion from II to III takes place from about 10° C. to about 20° C. In certain embodiments, the conversion from II to III takes place at about 10° C.

In certain embodiments, the conversion from II to III takes place between 0.5 hr and 10 hr. In certain embodiments, the conversion from II to III takes place between 1 hr and 5 hr. In certain embodiments, the conversion from II to III takes place in about 3 hr.

In certain embodiments, the conversion from III to V takes place from about 25° C. to about 100° C. In certain embodiments, the conversion from III to V takes place from about 40° C. to about 80° C. In certain embodiments, the conversion from III to V takes place from about 55° C. to about 60° C.

In certain embodiments, the conversion from III to V takes place between 0.5 hr and 10 hr. In certain embodiments, the conversion from III to V takes place between 1 hr and 5 hr. In certain embodiments, the conversion from III to V takes place in about 1 hr.

In certain embodiments, the conversion from V to IV takes place from about 20° C. to about 200° C. In certain embodiments, the conversion from V to IV takes place from about 50° C. to about 100° C. In certain embodiments, the conversion from V to IV takes place from about 85° C. to about 90° C.

In certain embodiments, the conversion from V to IV takes place between 0.5 hr and 10 hr. In certain embodiments, the conversion from V to IV takes place between 1 hr and 5 hr. In certain embodiments, the conversion from V to IV takes place in about 2 hr.

### 4. Novel Intermediates

The methods of the invention involve the generation and use of certain novel intermediate compounds. Novel intermediates of the invention include the following compounds of formula V:

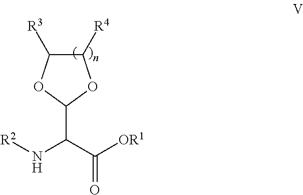

or a pharmaceutically acceptable salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
n is 0, 1, 2, or 3.

In certain embodiments, the invention provides a compound of formula V wherein R¹ is C₁₋₆ aliphatic which is optionally substituted. In certain embodiments, the invention provides a compound of formula V wherein R¹ is a 3-8 membered saturated or partially unsaturated carbocyclic ring which is optionally substituted. In certain embodiments, the invention provides a compound of formula V wherein R¹ is a 3-7 membered heterocylic ring having 1-4 heteroatoms independently selected from nitrogen, oxygen, or sulfur, which is optionally substituted. In certain embodiments, the invention provides a compound of formula V wherein R¹ is a 5-6 membered monocyclic heteroaryl ring having 1-4 heteroatoms independently selected from nitrogen, oxygen, or sulfur, which is optionally substituted.

In certain embodiments, R¹ is methyl, ethyl, propyl, i-propyl, butyl, s-butyl, t-butyl, straight or branched pentyl, or straight or branched hexyl; each of which is optionally substituted. In certain embodiments, R¹ is methyl or ethyl.

In certain embodiments, R¹ is phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, the invention provides a compound of formula V wherein R² is C₁₋₆ aliphatic which is optionally substituted. In certain embodiments, the invention provides a compound of formula V wherein R² is a 3-8 membered saturated or partially unsaturated carbocyclic ring which is optionally substituted. In certain embodiments, the invention provides a compound of formula V wherein R² is a 3-7 membered heterocylic ring having 1-4 heteroatoms independently selected from nitrogen, oxygen, or sulfur, which is optionally substituted. In certain embodiments, the invention provides a compound of formula V wherein R² is a 5-6 membered monocyclic heteroaryl ring having 1-4 heteroatoms independently selected from nitrogen, oxygen, or sulfur, which is optionally substituted.

In certain embodiments, R² is methyl, ethyl, propyl, i-propyl, butyl, s-butyl, t-butyl, straight or branched pentyl, or straight or branched hexyl; each of which is optionally substituted. In certain embodiments, R² is methyl or ethyl.

In certain embodiments, R² is phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, the invention provides a compound of formula V, wherein R² is —SO₂R¹. In certain embodiments, the invention provides a compound of formula V, wherein R² is —SOR¹. In certain embodiments, the invention provides a compound of formula V, wherein R² is —C(O)R¹. In certain embodiments, the invention provides a compound of formula V, wherein R² is —CO₂R¹. In certain embodiments, the invention provides a compound of formula V, wherein R² is —C(O)N(R¹)₂.

In various embodiments, the invention provides a compound of formula V wherein R³ is hydrogen.

In certain embodiments, the invention provides a compound of formula V wherein R³ is C₁₋₆ aliphatic which is optionally substituted. In certain embodiments, the invention provides a compound of formula V wherein R³ is a 3-8 membered saturated or partially unsaturated carbocyclic ring which is optionally substituted. In certain embodiments, the invention provides a compound of formula V wherein R³ is a 3-7 membered heterocylic ring having 1-4 heteroatoms independently selected from nitrogen, oxygen, or sulfur, which is optionally substituted. In certain embodiments, the invention provides a compound of formula V wherein R³ is a 5-6 membered monocyclic heteroaryl ring having 1-4 heteroatoms independently selected from nitrogen, oxygen, or sulfur, which is optionally substituted.

In certain embodiments, R³ is methyl, ethyl, propyl, i-propyl, butyl, s-butyl, t-butyl, straight or branched pentyl, or straight or branched hexyl; each of which is optionally substituted. In certain embodiments, R³ is methyl or ethyl.

In various embodiments, the invention provides a compound of formula V wherein R³ is halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In various embodiments, the invention provides a compound of formula V wherein R⁴ is hydrogen.

In certain embodiments, the invention provides a compound of formula V wherein R⁴ is C₁₋₆ aliphatic which is optionally substituted. In certain embodiments, the invention provides a compound of formula V wherein R⁴ is a 3-8 membered saturated or partially unsaturated carbocyclic ring which is optionally substituted. In certain embodiments, the invention provides a compound of formula V wherein R⁴ is a 3-7 membered heterocylic ring having 1-4 heteroatoms independently selected from nitrogen, oxygen, or sulfur, which is optionally substituted. In certain embodiments, the invention provides a compound of formula V wherein R⁴ is a 5-6 membered monocyclic heteroaryl ring having 1-4 heteroatoms independently selected from nitrogen, oxygen, or sulfur, which is optionally substituted.

In certain embodiments, R⁴ is methyl, ethyl, propyl, i-propyl, butyl, s-butyl, t-butyl, straight or branched pentyl, or straight or branched hexyl; each of which is optionally substituted. In certain embodiments, R⁴ is methyl or ethyl.

In various embodiments, the invention provides a compound of formula V wherein R⁴ is halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In various embodiments, the invention provides a compound of formula V wherein n is 1. In various embodiments, the invention provides a compound of formula V wherein n is 2.

In certain embodiments, the invention provides:

Methyl 2-(1,3-dioxolan-2-yl)-2-(methylamino)acetate:

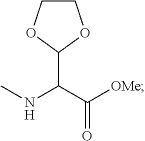

or
Ethyl 2-(1,3-dioxolan-2-yl)-2-(methylamino)acetate;

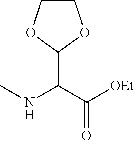

or a salt thereof.

### EXEMPLIFICATION OF THE INVENTION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in oC (degrees Centigrade). All reactions were conducted at room temperature unless otherwise noted.

All compounds of the present invention were synthesized by processes developed by the inventors.

¹H-NMR spectra were recorded on a Brooker Avance 400 MHz spectrometer of the Brooker Biospin GmbH. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), m (multiplet), or br (broad).

HPLC data was obtained using Agilent 1100 series HPLC from agilent technologies using an Column: YMC-Triart C18 3μ, 100×4.6 mm Solvent A: 950 ml of ammonium acetate/acetic acid buffer at pH=6+50 ml acetonitril; Solvent B: 200 ml of ammonium acetate/acetic acid buffer at pH=6+800 ml acetonitril; Flow: 1.5 ml/min; Gradient: 0 min: 5% B, 2 min: 5% B, 7 min: 20% B, 17 min: 85% B, 17.1 min: 5% B, 22 min: 5% B.

Some abbreviations that may appear in this application are as follows:

| | |
|---|---|
| δ | chemical shift |
| d | deuterium or doublet |
| dd | doublet of doublets |
| DCM | dichloromethane |
| DMF | dimethylformamide |
| DMSO | dimethylsulfoxide |
| THF | tetrhydrofuran |
| eq. | equivalent |
| h | hour |
| ¹H | proton |
| HPLC | high pressure liquid chromatography |
| J | coupling constant |
| LC | liquid chromatography |
| m | multiplet |
| M | molecular ion |
| MHz | Megahertz |
| min | minute |
| mL | milliliter |
| MS | mass spectrometry |
| m/z | mass-to-charge ratio |
| NMR | nuclear magnetic resonance |
| RBF | Round Bottom Flask |
| RT | room temperature |
| Rₜ | retention time |
| s | singlet |
| TLC | thin layer chromatography |
| UV | ultraviolet |

### Example 1

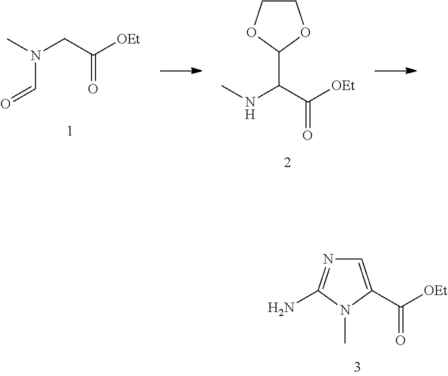

N-Formylsarcosine ethyl ester 1 (1.85 kg) was dissolved in toluene (3.9 kg) and ethyl formate (3.28 kg) and cooled to 10° C. A 20 wt-% solution of potassium tert-butoxide (1.84 kg) in tetrahydrofuran (7.4 kg) was added and stirring was continued for 3 h. The reaction mixture was extracted 2× with a solution of sodium chloride in water (10 wt-%) and the combined water extracts were washed 1× with toluene.

Aqueous hydrogen chloride (25% wt-%; 5.62 kg) was added to the aqueous solution, followed by ethylene glycol (2.36 kg). The reaction mixture was heated to 55-60° C. for 1 h before only the organic solvent residues were distilled off under vacuum.

Aqueous Cyanamide (50 wt-%, 2.16 kg) was then added at 20° C., followed by sodium acetate (3.04 kg). The resulting reaction mixture was heated to 85-90° C. for 2 h and cooled to 0-5° C. before a pH of ˜8-9 was adjusted via addition of aqueous sodium hydroxide (32% wt-%; 4.1 kg). Compound 3 (1.66 kg; 75%) was isolated after filtration and washing with water.

¹H-NMR (400 MHz, d6-DMSO): δ=1.24 (3H, t, J=7.1 Hz); 3.53 (3H, s); 4.16 (2H, q, J=7.0 Hz); 6.15 (s, 2H); 7.28 (s, 1H).

HPLC (R^{†}=7.7 min): 97.9% (a/a).

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of producing TH-302: 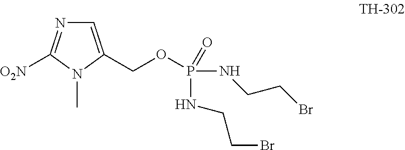 [SMILES: Cn1c(COP(=O)(NCCBr)NCCBr)cnc1[N+](=O)[O-]]
or a pharmaceutically acceptable salt thereof;
comprising the step of converting a compound of formula V 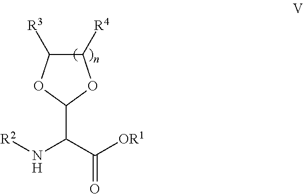
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
n is 0, 1, 2, or 3;
to a compound of formula IV 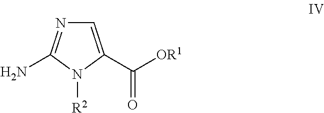
and converting the compound of formula IV to TH-302.

2. The method of claim 1, comprising the step of converting a compound of formula III 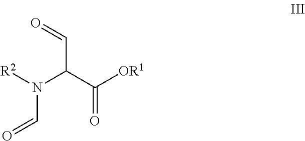
or enolate thereof,
to a compound of formula V

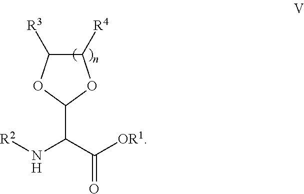

3. A method of producing TH-302: 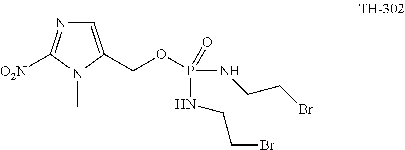 [SMILES: Cn1c(COP(=O)(NCCBr)NCCBr)cnc1[N+](=O)[O-]]
or a pharmaceutically acceptable salt thereof,
comprising the step of converting a compound of formula III 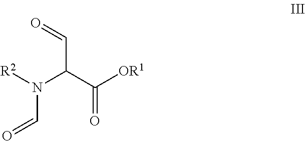
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
R² is —R¹, -haloalkyl, —SO₂R¹, —SOR¹, —C(O)R¹, —CO₂R¹, or —C(O)N(R¹)₂,
or enolate thereof,
to a compound of formula V 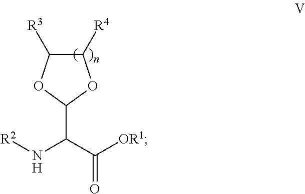
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
n is 0, 1, 2, or 3;
converting the compound of formula V to a compound of formula IV 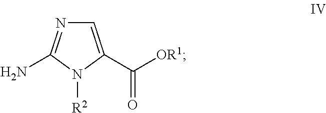
wherein
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
and converting the compound of formula IV to TH-302.

4. The method of claim 1, wherein each R¹ is independently C₁₋₆ aliphatic, which is optionally substituted.

5. The method of claim 4, wherein each R¹ is independently methyl or ethyl.

6. The method of claim 1, wherein each R² is independently C₁₋₆ aliphatic, which is optionally substituted.

7. The method of claim 6, wherein each R² is independently methyl or ethyl.

8. The method of claim 1, wherein III is produced by converting II to III, or enolate thereof, comprising the step of adding a base, a formyl source, and one or more solvents.

9. The method of claim 8, wherein the base is a metal hydroxide or an organic salt.

10. The method of claim 9, wherein the base is KOtBu.

11. The method of claim 8, wherein the one or more solvents are THF, Methyl-THF, toluene, xylene, ether, MTBE, cumene, aliphatic hydrocarbons or methylene chloride.

12. The method of claim 1, wherein the conversion of III, or enolate thereof, to V comprises an aqueous extraction of III in the presence of, or followed by addition of a water soluble diol, wherein V is in an aqueous layer.

13. The method of claim 12, wherein the diol is ethylene glycol.

14. The method of claim 12, further comprising the addition of a water soluble acid.

15. The method of claim 14, wherein the acid is HCl.

16. The method of claim 1, wherein the conversion of V to IV comprises a base and NC—NH₂.

17. The method of claim 16, wherein the base is NaOAc.

18. A compound of formula V: 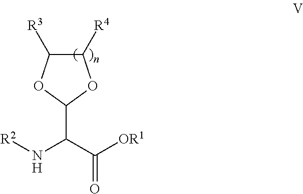
or a salt thereof,
wherein,
R¹ is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R¹, -haloalkyl, —SO₂R¹, —SOR¹, —C(O)R¹, —CO₂R¹, or —C(O)N(R¹)₂;
R³ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁴ is —R¹, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
n is 0, 1, 2, or 3.

19. The compound of claim 18, selected from:
Methyl 2-(1,3-dioxolan-2-yl)-2-(methylamino)acetate: 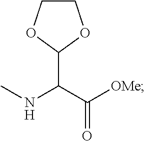
and
Ethyl 2-(1,3-dioxolan-2-yl)-2-(methylamino)acetate; 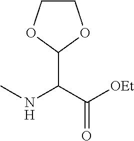
or a salt thereof.
